(19) United States
(12) Patent Application Publication
(10) Pub. No.: US 2014/0249618 A1
(43) Pub. Date: Sep. 4, 2014

(54) SITE SPECIFIC DRUG DELIVERY WRAPS, SYSTEMS AND METHODS OF USE THEREOF

(71) Applicant: Elutin, Inc., Las Vegas, NV (US)

(72) Inventor: David S. Cohen, Las Vegas, NV (US)

(21) Appl. No.: 14/181,353

(22) Filed: Feb. 14, 2014

Related U.S. Application Data
(60) Provisional application No. 61/765,842, filed on Feb. 18, 2013.

(51) Int. Cl. A61B 17/12

(52) U.S. Cl. CPC A61B 17/12022
USPC 623/1.15

(57) ABSTRACT

This invention is directed to systems for the treatment of vasculature stenosis having an interior (adventitial) and exterior side (interstitial), wherein the interior and exterior sides are biodegradable and the interior side has an adhesive flexible, biodegradable matrix material and an antiproliferative agent, wherein the anti-proliferative agent is released from the matrix substantially following a unidirectional flow pattern towards the perivascular region, exerting biological activity on the vascular smooth muscle cells of the vascular wall and the size of the system can be customized for adhering to a vascular site of interest.

### RELATED APPLICATIONS

This application claims priority under 35 U.S.C. §119(e) from Provisional U.S. Patent Application Ser. No. 61/765,842 filed on Feb. 18, 2013. The entire contents of each of which are herein incorporated by reference.

### FIELD OF THE INVENTION

This invention relates to medical methods, kits, and systems for delivery of at least one therapeutic agent to a site of interest for improving and maintaining the integrity of vessels, passageways, tubes or bodily cavities in subjects at risk of tissue hyperproliferation that may result in reductions or complete blockage of flow of biological fluids such as blood, lymph, and cerebral spinal fluid.

### BACKGROUND OF THE INVENTION

Vascular diseases such as peripheral artery disease, cerebral artery disease, coronary artery disease, blood disorders that affect circulation, renal artery disease, varicose disease, as well as vascular procedures to ameliorate reductions in tissue perfusion such as coronary artery bypass grafting (CABG), percutaneous transluminal angioplasty (PTA), carotid endarterectomy and bypass grafting (autogenous vein or synthetic graft) of the femoral, tibial, fibula (peroneal), iliac, ulnar, radial, brachial, coronary and renal arteries are some of the conditions that can lead to undesired local vascular tissue hyper-proliferation and a high recurrence of re-occlusion.

At the vascular level, intimal hyperplasia is a common response to vessel injury or a bypass graft failure at the site of anastomosis. Such an undesired response will eventually lead to the narrowing of the blood vessel and subsequently compromise the integrity of blood vessels in patients suffering from such conditions. Despite many available medical modalities currently available to limit the degree of tissue hyper-proliferation, there are still limitations in addressing the need of patients at risk of such a condition.

For example, patients undergoing successful CABG or PTA procedures are at risk or have an elevated incidence of restenosis at the site of the respective medical intervention. Pharmacological attempts to reduce the incidence of restenosis have largely been unsuccessful. Devices that are typically used today to prevent restenosis, although in most part successful, have their own specific limitations. Such devices are intravascular and are either in the form of a bare metal or drug-eluting stent (DES) or a balloon catheter or drug-eluting balloon (DEB). Aside from the potential risk of thrombosis and malapposition to the vessel walls as is seen with stents, these type of devices in peripheral arterial disease applications have failed to demonstrate long-term clinical efficacy superior to those observed with simple balloon angioplasty and bare metal stents.

In hemodialysis patients, polytetrafluoroethylene (PTFE) grafts and arterial-venous fistulas are currently employed as access ports for dialysis filtration of blood in patients awaiting a kidney transplant. Although these modalities have been used successfully for many years, many fail for a variety of reasons including a high incidence of occlusion at the venous-graft/fistula anastomosis. Consequently, they lose their utility in hemodialysis patients.

The present invention addresses the problem associated with the existing procedures and offers significant advantages over the current ‘state of the art’ pharmacological and intravascular treatment of undesired tissue hyper-proliferation.

### SUMMARY OF THE INVENTION

The teachings herein are generally directed to novel drug-eluting systems for treating subjects at risk of undesired tissue (i.e. the coronary, cerebral, and peripheral vasculature, esophagus, trachea, bronchi, alveoli, colon, biliary tract, urinary tract, prostate, brain, and the like) hyperproliferation. In one embodiment the drug eluting systems of the present invention prevent or delay a blockage or a narrowing of a tubular structure within a subject's body.

In another aspect, the present invention is directed to methods of treating restenosis at a site that is at risk of developing vasculature blockage. In one embodiment, a drug eluting system is described that comprise an adhesive, biocompatible, biodegradable matrix customized for application to an anatomic region of interest.

Another aspect of the present invention is directed to an implantable, antiproliferative agent-administering perivascular system adapted to be placed in contact with the exterior side of a vascular structure. According to this aspect, the perivascular system has an adventitial side (interior) and an interstitial side (exterior), wherein the adventitial side contains an adhesive flexible, biodegradable matrix material and at least one antiproliferative agent. In at least one embodiment, the antiproliferative agent is released from the matrix in a unidirectional flow pattern into the vascular wall of the vascular structure suffusing all vascular layers from the adventitia to the intima. In one embodiment, the size of the system can be customized for adhering to a vascular site of interest and be administered for a sufficient duration of time until an optimal clinical outcome is observed.

In another aspect of this invention, the described implantable drug eluting system is in the form of a wrap such as a vascular wrap, a film, a sheet, a tube, a mesh, a sheath or even a biodegradable cloth. In another embodiment, the presently described system is formed of matrix material containing an oily component, optionally a natural or synthetic polymer such as chitosan, alginate, polyethylene glycol, polylysine, hyaluronic acid, chondroitin sulfate, collagen, omega-3 and omega-6 fatty acids, tocopherols, or mixtures thereof and at least one therapeutic agent.

In another aspect of the present invention, the drug eluting system is degraded and absorbed within six (6) months after it is implanted or placed inside the body of a subject in need of such treatment.

In a preferred embodiment, the system has an adventitial and an interstitial side, wherein the adventitial side comes into direct contact with the external wall of the passageway, vessel or body cavity of interest and the interstitial side of the system is positioned facing the interstitial space (interstitium). In one embodiment, the matrix material that comes into contact with the external side of a passageway, a vessel or a body cavity wall, is of appropriate elasticity and tensile strength to allow complete adhesion to the external wall of the vessel for a desired period of time without decreasing flow.

### BRIEF DESCRIPTION OF THE DRAWINGS

FIG. 1 is a cross-section of a single sheath embodiment of the present invention;

FIG. 2 is a cross-section of a multiple overlapping sheath embodiment of the present invention;

FIG. 3 is a schematic depiction of an arterial wrap covering the graft in coronary artery bypass graft surgery;

FIG. 4 is a schematic of an overlapping drug eluting mesh wrap at the anastomotic arterial bypass site; and

FIG. 5 is a schematic of a multilayer drug-eluting biodegradable vascular wrap.

### DETAILED DESCRIPTION OF THE INVENTION

### Definitions

As used herein, the terms “Adventitial side,” proximal or interior side may be used interchangeably and refer to the side of the system that comes in contact with the outermost connective tissue covering of any vessel, or other structure.

As used herein, the terms “Interstitial side” distal or exterior side may be used interchangeably and refer to the side of the system that comes in contact with the space and elements thereof surrounding a given tissue or organ. It may be filed with interstitial fluid or be a small opening or space between organs or tissues.

As used herein, “Matrix Materials” are the material used for the matrix that may be from natural sources or may be synthetically manufactured or both. For example, a system according to the present invention may employ a biocompatible, biodegradable, bioabsorbable substance such as a fatty acid derivative, poly(l) lactic acid, fibrin, hyaluronic acid, collagen, omega-3 fatty acids, omega-6 fatty acids, tocopherols, or chitosan. A suitably biocompatible, biodegradable Matrix Material may also contain two or more biodegradable substances. Other factors to be considered in the selection of a particular Matrix Material include the viscosity, thickness, porosity of the material, drug loading capability and controllability of its rate of drug release and biodegradation. The characteristics of the matrix material afford the system a net unidirectional flow of the therapeutic agent(s) towards the site of interest.

As used herein, the term “biocompatible polymer” refers to polymers which, in sufficient amounts are non-toxic, chemically inert, and substantially non-immunogenic when used internally in the subject. Suitable biocompatible polymers include, by way of example, poly-1 or d-lysine, chitosan, alginate, poly lactic acid, poly glycolic acid, copolymers of poly lactic and poly glycolic acid, polycaprolactone, polyhydroxybutyrate valerate, fibrin, collagen, omega-3 fatty acids, omega-6 fatty acids, tocopherols, 3-(glycidyloxypropyl) triethoxysilane, Magnesium (Mg) alloys, polyketal-poly(cyclohexane-1,4-diyl acetone dimethylene ketal), polyethylene glycol-polylactic acid (PEGPLA). Preferably, the biocompatible polymer does not induce chronic inflammation when employed in vivo or at a vascular site; it is biodegradable and will completely degrade within six (6) months after placement or administration in vivo.

As used herein the term “net unidirectional flow” is meant to convey that the majority of the therapeutic agent embedded or present in the Matrix Material is delivered towards the site of interest. In the preferred embodiment, the net unidirectional flow provides at least 85%, 90%, 95%, 97%, 99% of the therapeutic agent towards the site of interest during the treatment period.

The characteristics of the Matrix Material with respect to thickness, porosity, viscosity, rate of biodegradation need not be identical throughout the matrix. It is contemplated that by creating a suitable mixture of ingredients, the matrix material possess adhesive properties suitable to ensure complete adhesion of the system to the external vasculature wall without constricting the vessel and reducing flow, so that formation of a reservoir or space susceptible to microbial contamination during the course of treatment is minimized.

As used herein “hyperproliferative condition” refers to undesirable tissue growth associated for example with such conditions as atherosclerosis and restenosis.

The phrase “site of interest” as described herein refers to passageways, tubes or cavities, vessels, that might have been subject to a surgical procedure, percutaneous transluminal angioplasty, bare metal stent implantation, or are at risk for developing a hyperproliferative condition such as restenosis.

As used herein the term “subject” is a mammal, including human and a non-human mammal.

As used herein the term “substantially free” means a condition whereby the ingredient listed is below 0.25% of the total weight of the product.

As used herein the term “treatment” does not refer to an absolute cure, but includes prophylactic, preventative or chronic treatment for achieving optimal clinical outcome.

As used herein a “wrap” includes a layer of coating, a mesh, a cloth, a sheet, or a sheath that may be covering or be wrapped, either completely or partially, around an anatomical site of interest such as a passageway, a vessel, a body cavity, a body tube, or the like by winding, folding, or suturing to the adventitial or external side of the site of interest thereby maintaining effective amounts of a therapeutic agent in the region in need of the treatment.

### ASPECTS OF THE INVENTION

Generally speaking the present invention is directed to novel drug-eluting systems for treating areas in a subject at risk of undesired tissue hyperproliferation. Such areas include for example the coronary, peripheral, and cerebral vasculature, esophagus, trachea, colon, biliary tract, urinary tract, prostate, and the like.

In one embodiment the drug eluting systems of the present invention reverse or treat a blockage or a narrowing of a tubular structure within a subject's body. At least one aspect of the present invention is directed to drug eluting systems for wrapping perivascularly at a site of interest for delivery of a therapeutic agent. In a preferred embodiment such a perivascular system provides a unidirectional flow of the therapeutic agent embedded or incorporated into the matrix material. In at least another embodiment, the systems of the present inventions are customized to prevent, inhibit, suppress and treat vascular smooth muscle cell proliferation at a site at risk of tissue hyperproliferation. In a more preferred embodiment, the invention is also directed to methods of providing a net unidirectional flow of the therapeutic agent with at least 85% of such agent being delivered to the site of interest, thereby combating local cellular or tissue hyperproliferation.

In at least one aspect of the present invention, the Matrix Material contains a biodegradable polymer or substance. Examples of biodegradable polymers or substances which can be used in this invention include polymers or copolymers containing omega-3 or omega-6 fatty acids, vitamin E, polylysine, polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, chitin, chitosan, gelatin, collagen, hyaluronic acid, fibrin, and copolymers, or mixtures of the aforementioned materials.

Other biodegradable material include polyglycol acid, poly (epsilon caprolactone), poly (diol citrates), polycaprolactone-polylactic acid, Tepha-Flex, polyhydroxybutyrate valerate, fibrin, collagen, omega-3 fatty acids, omega-6 fatty acids, tocopherols, 3-(glycidyloxypropyl) triethoxysilane, polyketal-poly(cyclohexane-1,4-diyl acetone dimethylene ketal), polyethylene glycol-polylactic acid (PEG PLA), magnesium alloy (93%), tyrosine-based and fibrinogen-collagen complex. As provided herein the actual, definitive and specific properties of the material(s) of the Adventitial and Interstitial sides of the system depends on the intended clinical use of the system (e.g. critical limb ischemia, oncology, ophthalmic, A-V access/fistula, CABG) and the therapeutic agents or biological mediators it will release for the specific site of interest. In the most preferred embodiment, the system is completely biodegraded over a period of 4-12 months.

In a preferred embodiment, these polymers and copolymers are biocompatible. Preferred biocompatible polymers include polymers with at least one fatty acid moeity such as omega-3 or omega-6 fatty acid, polylactides, polycaprolactones, polyglycolides, polyethylene glycol and copolymers thereof having base units of omega-3 or omega-6 fatty acid, glycolide, lactide, caprolactone and polyethylene glycol. The more preferred embodiment can possess a suitable number of amorphous regions to enhance porosity. Lower molecular weight polymers are more suitable for desired biodegradation. The higher molecular-weight polymers tend to coagulate and stay intact for a longer period of time. A suitable polymer of the present invention may include a blend of lower and higher molecular weight polymers.

In one embodiment, the Matrix Material includes an oily component having polymers or copolymers containing omega-3 and/or omega-6 fatty acid moieties. Such moieties include auto-polymerized omega-3 or omega-6 fatty acid, fish oil fatty acid, free fatty acid, triglycerides, esters thereof alone or in combination. Fish oil fatty acid of choice include one or more of arachidic acid, gadoleic acid, arachidonic acid, eicosapentaenoic acid, docosahexaenoic acid or derivatives, analogs and pharmaceutically acceptable salts thereof. The free fatty acid can include one or more of butyric acid, caproic acid, caprylic acid, capric acid, lauric acid, myristic acid, palmitic acid, palmitoleic acid, stearic acid, oleic acid, vaccenic acid, linoleic acid, alpha-linolenic acid, gamma-linolenic acid, behenic acid, erucic acid, lignoceric acid, and pharmaceutically acceptable salts thereof.

In another embodiment, the Matrix Material includes a vitamin E component which can also be combined with the oily component. The vitamin E component can be any one or more of the compounds such as alpha-tocopherol, beta-tocopherol, delta-tocopherol, gamma-tocopherol, alpha-tocotrienol, beta-tocotrienol, delta-tocotrienol, gamma-tocotrienol, alpha-tocopherol acetate, beta-tocopherol acetate, gamma-tocopherol acetate, delta-tocopherol acetate, alpha-tocotrienol acetate, beta-tocotrienol acetate, delta-tocotrienol acetate, gamma-tocotrienol acetate, alpha-tocopherol succinate, beta-tocopherol succinate, gamma-tocopherol succinate, delta-tocopherol succinate, alpha-tocotrienol succinate, beta tocotrienol succinate, delta-tocotrienol succinate, gamma-tocotrienol succinate, mixed tocopherols, and pharmaceutically acceptable salts thereof. In these embodiments, the omega-3 or omega 6, the fish oil or the vitamin E component may be used in concentrations ranging from about 2% to 95% by weight, based on the total weight of the final product. In another embodiment, suitable concentrations of chitosan can be in the range of from about 2% to about 75% by weight of the final product. In aspects of the invention, the chitosan concentrations are in the range from about 5% to 65% or in the ranges of 20% to about 55% by weight.

In at least one embodiment, the Matrix Material includes chitosan. Chitosan is a natural biopolymer with favorable biocompatibility, biodegradability, hemostatic activity, and anti-infective activity. In such an embodiment, chitosan may be used in the invention in concentrations ranging from about 2% to 95% by weight, based on the total weight of the final product. In another embodiment, suitable concentrations of chitosan can be in the range of from about 5% to about 75% by weight of the final product. In aspects of the invention, the chitosan concentrations are in the range from about 20% to about 65% by weight. However one of skill in the art would understand that any sub-ranges are contemplated by the present invention.

In another embodiment, the Matrix Material includes gelatin. Gelatin of the present invention is biodegradable, biocompatible, and essentially non-immunogenic. Gelatin for use in the present invention includes both Type A or Type B, or combinations thereof. In such embodiment, gelatin may be used in concentrations from about 2% to 95% by weight, based on the total weight of the product. In another embodiment, gelatin may be used in the invention in concentrations ranging from about 5% to 75% by weight, based on the total weight of the final product. In aspects of the invention, the gelatin concentrations are in the range from about 20 to about 65% by weight. However one of skill in the art would understand that any sub-ranges are contemplated by the present invention.

In another embodiment, the Matrix Material includes an oily component, chitosan, gelatin and at least one other biodegradable, biocompatible polymer, the concentrations of which are adjusted to maximize drug load for the therapeutic outcome sought.

At least one aspect of the present invention is to minimize the risk of developing a post-surgical infection at the site of the presently described drug-eluting system as compared to commercially available wraps used for the same intended purpose. In at least one embodiment, the Material Matrix is completely free or substantially free of PTFE. PTFE is non-biodegradable and as such if used, it will remain resident at the site of application. The presence of the PTFE wrap creates a perivascular space that can turn into a reservoir for microbial contamination resulting in local infections and/or systemic sepsis. This invention being completely biodegradable minimizes the risk of developing a perivascular space between it and the adventitial side of the vessel consequently avoiding bacterial contamination

In one aspect of the present invention, the polymeric matrix and the fatty acid component are cured to increase viscosity to form the desirable shape. The curing methodologies are known in the art and include application of UV light, application of heat, airflow, and reaction with a gas or chemical cross-linker. In another aspect of the present invention, the polymeric matrix is sterilized by ethylene oxide, E Beam or gamma ray sterilization to render it acceptable for medical use.

In the preferred embodiment, the drug eluting system include at least one therapeutic agent selected from the group of antiproliferatives, chemotherapeutic agents, anti-thrombotic agents, antioxidants, anti-inflammatory agents, anesthetic agents, anti-infective agents, anti-inflammatory compounds, tissue absorption enhancers, analgesics, antiseptics, antibiotics, antithrombotic agents, estrogen analogues and other suitable agents. Such agents can be dispersed, embedded, encapsulated, bound or suspended within the Matrix Material.

In one embodiment, the anti-inflammatory is selected from the group of compounds including corticosteroid such as hydrocortisone, prednisolone, triamcinolone, dexamethasone, or a non-steroidal anti-inflammatory such as aspirin, ibuprofen, naproxen, diclofenac, or combinations thereof.

In another embodiment, the antiplatelet agents that can be employed in this system are selected from the following group: aspirin, P2Y12 receptor antagonists such as ticlopidine, clopidogrel, elinogrel, prasugrel, and ticagrelor; glycoprotein IIB/IIIA antagonists such as abciximab; antithrombins such as the factor 10a inhibitors rivaroxaban, apixaban, and fondaparinux; the direct thrombin inhibitors ximelogatran and dabigatran, anticoagulants including unfractionated and fractionated heparin and warfarin; calcium channel blockers such as diltiazem, nifedipine, amlodipine, etc; angiotensin receptor antagonists such as valsartan, irbesartan, candesartan, etc.; angiotensin-converting enzyme inhibitors such as captopril, enalapril, lisinopril, benazepril, etc.; protease inhibitors; phosphodiesterase inhibitors such as sildenafil, vardenafil, and tadalafil; and ethanol. The antiseptic can be any of the following: silver; iodine; phenols; terpenes; triarylmethane dyes; quaternary ammonium compounds such as chlorhexidine, polyhexamethylene biguanide, benzalkonium chloride; combinations thereof.

In another embodiment, the antibiotic of choice may include any single or combination of active agents that can prevent or treat infections secondary to gram negative, anaerobic or gram positive bacterial sources. Such agents include aminoglycosides such as amikacin, gentamicin, kanamycin, tobramycin, B-lactams such as penicillin, piperacillin, cephalexin, cefazolin, cefixime, imipenem, or quinolones such as ciprofloxacin, moxifloxacin, trovafloxacin, or clindamycin, vancomycin, rifampin, minocycline, or combinations thereof.

In at least one embodiment, the therapeutically active agents are dispersed, adsorbed, embedded, suspended, encapsulated in or layered on the Matrix Material to provide optimal rate of delivery. In an alternative embodiment, the therapeutically active agent can be administered alone or in combination with other agents. In another embodiment, depending on the therapeutic agent used in the combination within the matrix, the concentration of the therapeutic agent(s) will not be the same throughout the entire matrix.

In at least one embodiment, the drug eluting system is a unidirectional unilayer wrap containing Matrix Material having an omega-3 or omega-6 fatty acid component and an antiproliferative agent. In an alternative embodiment, the system may include additional coatings disposed on the interior side of the wrap.

In another embodiment, the drug-eluting system of the present invention would provide continuous delivery and adhesion to the site of interest for three (3) to twelve (12) months. Suitable agents to increase the adhesive properties include isocyanate substituted polyalkylene oxide, Photosensitive macromers such as polyethers and acrylates, Mytilus galloprovincialis foot protein-5 and a recombinant protein derived from mussels, Acrylate modified, natural fatty acid based hybride polymers, Mucilaginous polysaccharide/starch composite gel, Alpha-glucan aldehyde and an amino-group-containing polymer, Modified collagen, phosphoester polyamines, Modified chitosan, Isocyanate-functionalized polysaccharides, Degradable polyesters, such as polycaprolactone diol or polylactic acid, Thermoplastic lactide-containing polymers, Polylactides, polyglycolides, polylactones, polyorthoesters, polyanhydrides, polyglycolic acid, polycaprolactone, Amylopectin, and isocyanate-capped polymeric polyether-polyol.

The implantable drug-eluting system of the present invention is free or substantially free of PTFE but capable of being directly implanted to the site of anastomosis as such minimizing the subjects' risk of developing post-surgical infection or sepsis. The PTFE free aspect of the present invention prevents the system to act as a reservoir or depot for undesirable post-surgical exudates that may travel into the system.

In another aspect of the invention, the degradation rate of the Material Matrix can be adjusted by altering the ratio of the oily component, and the polymers such as an omega-3 or omega-6 fatty acid component, gelatin to collagen to chitosan. In another aspect of the invention, the system is in a suitable shape for extravascular wrapping of a site of interest. Preferred shapes for extravascular wrapping include cylindrical wrap, shealth, film, gel, foam or mesh.

In at least one aspect of the invention, the system contains at least one antiproliferative agent that will be released in a unidirectional fashion from the system into the vascular wall. In this embodiment, the antiproliferative agent prevents, suppresses or treats the smooth muscle proliferative response that contributes to the underlying pathophysiology of the restenosis, neointimal hyperplasia and narrowing of the vessel of interest. Such agents include but are not limited to sirolimus and analogs thereof such as everolimus, biolims, novolimus, etc., paclitaxel and other taxanes, tacrolimus, angiopeptin, vassenoids, flavoperidol, hormones such as estrogen, halofuginone, fondaparinux, matrix metalloproteinase inhibitors, ribosimes, interferons, antisense oligonucleotides and functional analogues thereof that produce comparable antiproliferative properties as their parent compound, angiotensin converting enzyme inhibitors, angiotensin receptor antagonists, phosphodiesterase inhibitors, iloprost and other analogs of prostacyclin and prostaglandin E₂, nitric oxide donors such as sodium nitroprusside, nitroglycerin, and isosorbide dinitrate, adenylate and guanylate cyclase stimulators, protease-activated receptor (PAR) 1 and 4 antagonists, and direct thrombin inhibitors. In a preferred embodiment, the antiproliferative agent is paclitaxel or rapamycin impregnated, absorbed, saturated, dispersed or immobilized with the Matrix Material. In at least one embodiment, the antiproliferative agent is rapamycin in amounts of about one to three (1-3) micrograms/mm2 (Range 1 micrograms-200 micrograms/mm2). In another embodiment, the antiproliferative agent is paclitaxel in amounts of about 0.9-2.6 micrograms/mm2 (Range 0.9-100 micrograms/mm2).

In at least one aspect of the invention, the combination of active ingredients can be customized based on the patient's needs and medical conditions. For example the system for a subject with a history of diabetes may differ in the combination of active ingredients and delivery rate as compared to a subject that is non-diabetic.

Other additive materials may be included in the system to provide the desired flexible properties, porosity, viscosity, and delivery and degradation rate. Examples of such materials include polyethylene glycol (PEG), glycerol, propylene glycol, dibutyl phthalate, triacetin, triethyl citrate, water, alcohol, vegetable oil, organic solvents, and the like. Such materials may be incorporated in any amount that does not adversely affect the dissolution rate of the final product or compromise the biocompatibility and biodegradability of the system and the release kinetics of the elutant as it may be understood by one of skill in the art.

The systems of the present invention may be prepared by suitable methods of making but include the steps of mixing the ingredients of the Matrix Material adding the therapeutically active agent(s) together to form a composition that may be molded or cured into the desired shape. Such a process may further include an appropriate process of sterilization of the final product.

In yet another aspect of the present invention, the Matrix Material can further include a plurality of particles. In one embodiment, the particles can include an active therapeutic agent or a blend of the active therapeutic agent in a polymer or a phospholipid moiety. The size of the particles can be selected to facilitate encapsulation or entrapment of the therapeutic agent. In particular, the sizes can be in nanometer to micrometer ranges. In at least one embodiment, the particles may be a nanoparticle, a microparticle, a liposome or a micelle that are dispersed, embedded, bound or layered in the Matrix Material. In another embodiment, the average particle size can range from 5 to 30,000 nm. In another embodiment, the average particles size can be less than 25 nm, 50 nm or 100 nm, between 100-400 nm, between 100-5000 nm, or greater than 5000 nm.

In at least one embodiment, the average molecular weight of the polymer is from about 1,000 daltons to about 500,000 daltons, more preferably from about 10,000 daltons to about 250,000 daltons and still more preferably from about 25,000 daltons to 150,000 daltons. As is apparent to one skilled in the art, adjustment of the viscosity of the composition can be readily achieved by mere adjustment of the molecular weight of the polymer composition.

In at least one embodiment, the Matrix Material contains omega-3 fatty acid compounds that are manufactured into a sheet, sleeve, shealth or mesh. In another embodiment the same is in manufactured in a cylindrical or tubular shape or any other geometrical shapes that may be customized based on the subjects medical needs. In a more preferred embodiment, the system is directly adhered to the outside of the native vessel of interest, at the site of graft anastamosis and/or over the vein, artery or graft itself or over arteries having undergone prior or concomitant balloon angioplasty with or without bare metal stent implantation.

At least one aspect of the invention provides that the system stay adhered to the outside of the native vessel throughout the entire treatment period of up to twelve (12) months, preferably up to six (6) months or at least four (4) months, without absorbing any local exudate or act as a reservoir for infective sources. In one embodiment, the system stays intact for at least four (4) months. In this aspect, the system allows for permeation of the chosen antiproliferative agent through the vessel wall at a rate for drug to eliminate, inhibit, prevent or treat the restenosis or hyperpoliferative behavior of the internal vessel lumen at risk of narrowing or blockage. In a more preferred embodiment, Matrix Material continues to be adhered to the vessel wall until the system is completely resorbed.

In another embodiment, the invention is directed to kits containing the drug eluting system for improving and maintaining the integrity of passageways, tubes or cavities following intrusion including a degradable sheath delivering at least one active component. The kit can further contain active components of diverse mechanisms of action for improving and maintaining the integrity of body passageways, tubes or cavities such as catheters, tubing, sutures or other material needed for cardiovascular surgery or balloon angioplasty. In this embodiment, the medical system is fabricated to be used in a fully biodegradable mesh, sheet, film, foam, gel or coating formulation, to wrap around the surgically implanted and/or dilated blood conduit vessel.

In such a scenario the system will contain effective concentrations of one or more of the active components that are vascular smooth muscle cell proliferation inhibitors to be released over an extended period of time. Accordingly, at least one aspect of the present invention is to provide kits for accompanying all surgically bypassed and/or balloon dilated blood vessels (with or without bare metal stent implantation) with overlapping biodegradable mesh, sheaths, or sheets to be wrapped around each artery or anastomotic site. The wrap will contain one or more anti-proliferative agents to be released adventitially for blocking or inhibiting vascular smooth muscle cell proliferation thereby improving the patient's standard of care.

In another embodiment, the invention is directed to a drug eluting system placed around an internal body organ, tissue or other internal surfaces to cover the site of interest preferably for delivering one or more drugs or biological agents of diverse mechanisms of action and for treatment of a designated body portion which has been damaged or intruded by surgery.

In another aspect of the present invention, the inventor describe methods of inhibiting stenosis of hemodialysis access grafts by placing the presently described system above, over or around a graft or vascular structure and/or at the site of anastomosis, maintaining the site adhesion until effectively inhibiting stenosis of the area of interest.

In yet another aspect of the present invention, the implantable drug eluting system is an absorbable wrap that may be placed in open bodily spaces. In this aspect of the invention, the system placed post-operatively between internal body tissues that have been separated by a surgical procedure. In this embodiment, the Matrix Material of the wrap is of biocompatible and biodegradable materials with at least one anti-proliferative agent either embedded or dispersed in the outermost layer of the matrix. In a more preferred embodiment, the Matrix Material further possess adhesive properties in the innermost part of the system intended to hold the wrap to the outside vessel wall. In yet a more preferred embodiment, the antiproliferative agent is in nanoparticle form having a dimension between 25-400 nm.

In yet another aspect of the present invention, methods of treating and inhibiting restenosis are described. In accordance to this aspect of the invention, anti-proliferative agents contained in implantable meshes, sheets, or sheaths are placed between two adjacent tissues of a subject in need of such treatment. Accordingly, the mesh, sheath, or sheet would be inserted during a surgical procedure before closing of the surgical incision at the site of interest to remain for at least a period of up to about six (6) months. When this system is absorbed into the body, the anti-proliferative drug attached to the material (i.e. mesh, sheath, sheet, etc.) is dispersed and delivered in a unidirectional fashion, into the site of interest.

In this embodiment, a mesh, sheet or sheath is wrapped around the perivascular region of a vessel with an arterial-venous anastomosis. In such a scenario, the antiproliferative agent permeates out of the wrap into the vessel wall and thus into the region at risk of stenosis (the venous end of the anastomosis). In at least one embodiment, the mesh, sheet or sheath is rolled into a cylinder and placed into a generally cylindrical cavity of the human body that has undergone a surgical procedure. The mesh, sheath or sheet, could also be placed around a vascular graft or even a permanent system that resides in the human body.

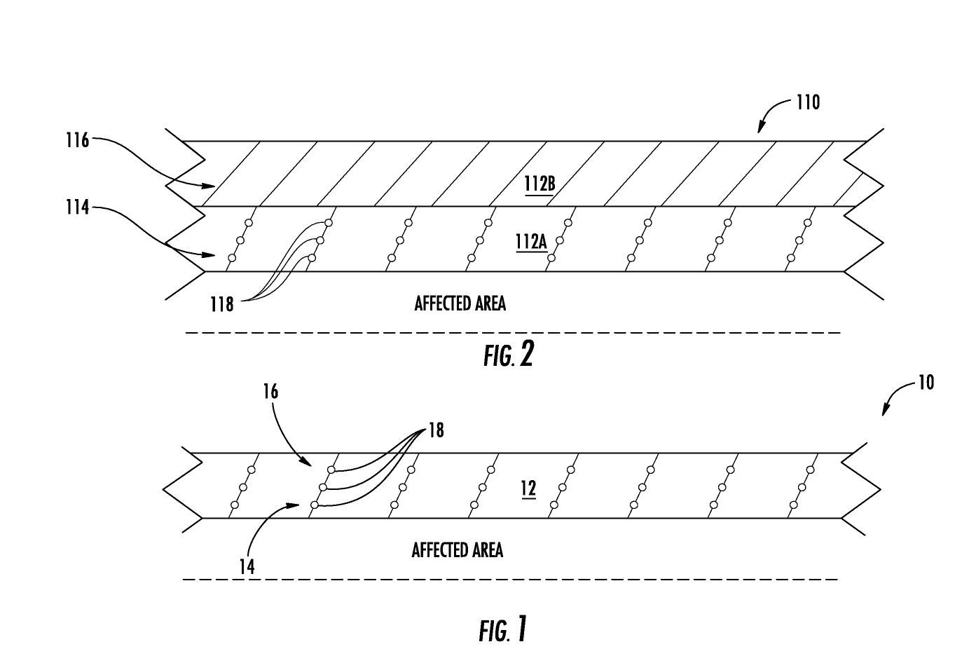

Figuratively speaking, at least one embodiment of the present invention is the system 10 which includes a surgical wrap 12 having an Adventitial (interior) side 14 and an Interstitial (exterior) side 16 wherein the proximal (interior) side 14 has at least one therapeutic agent component 18 and comes into direct contact with the exterior wall of a vessel. See FIG. 1.

In another embodiment, the therapeutic agent is dispersed uniformly in the adventitial side of the system when in contact with the site of interest. In another embodiment, the interstitial side of the system may have a coating that facilitates the unidirectional flow pattern for delivery of a therapeutic agent to the site of interest. In at least one embodiment, the exterior coating will be inert and not reactive to interstitial components to which it comes in contact.

In a preferred embodiment, the direction of the flow of the therapeutic agent is from the Interstitial (outmost exterior) side of the wrap to the adventitial side internally, inward toward the sites of interest within the walls of the treated blood vessel or anastomotic bypass segments. In this case, the system is designed to be a concentric circle around the site of interest, the interstitial side will secure the active component and the adventitial side would adhere to the affected area thereby facilitating the permeation of the therapeutic agent from the system into the vascular wall.

In at least one embodiment, the wrap and the active component of the Adventitial side degrade at different rates. This embodiment, allows the Interstitial side of the wrap to act as a seal or “cap” to ensure the active component flows in the interior direction or adventially. As with the active component of the interior side, the selection of the outmost exterior side's coating will be defined by the required use and environment wherein the system is to be used.

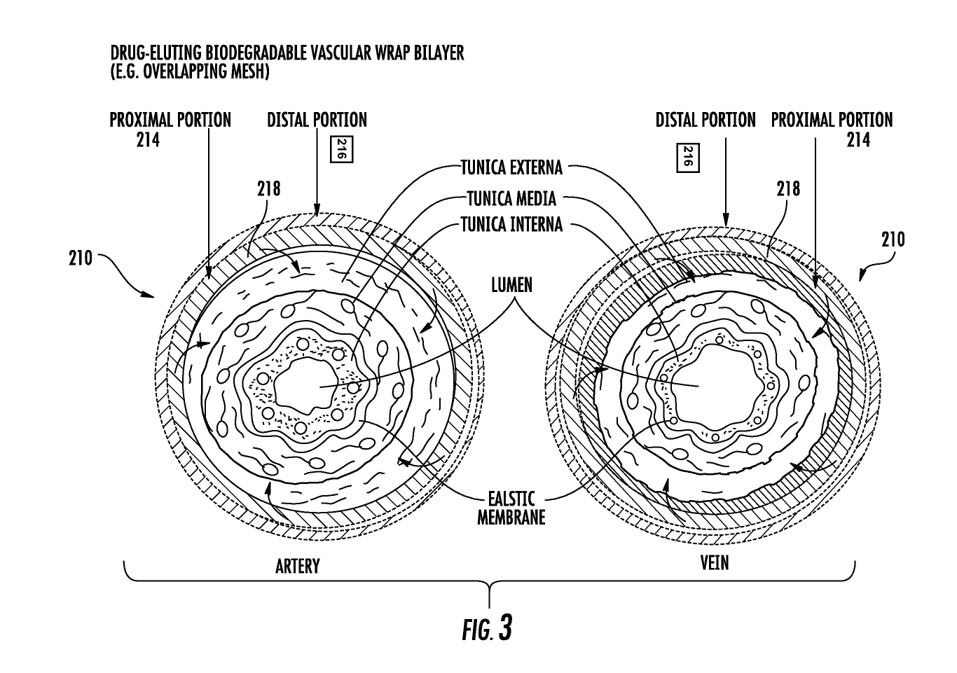

In another embodiment, FIG. 2, the system 110 contains two overlapping sheaths 112A, 112B, wherein the active ingredient 118 is on the Adventitial (interior) side 114 and the Interstitial (exterior) side 116 is of a designated material as in the interstitial side 16 of the previous embodiment. In another embodiment, the concentration of the therapeutic agent contained in the Matrix Material of the Interstitial side is higher than the Adventitial side by at least 5%, 10%, 20%, 30%, 40%, 50%, 60%, 70%, 80%, 85%, 90%, 95%, 98%. The use of multiple layers of adhering sheaths further secures the interior portion to the affected area supporting the “one-way” flow.

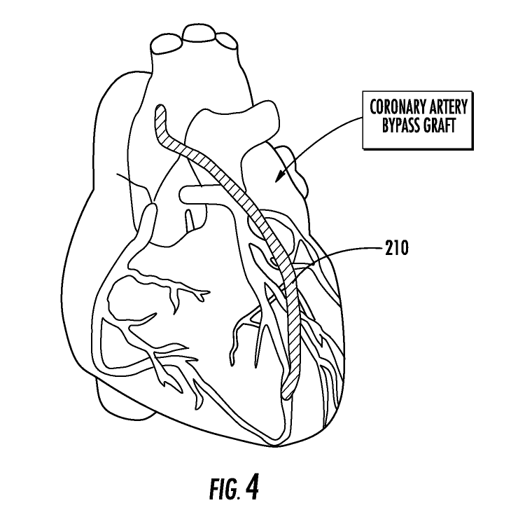

FIGS. 3, 4 and 5 are directed to alternative embodiments. FIG. 3 is the illustration of the present invention 210 as an adhering biodegradable/bioabsorbable bilayer vascular wrap for improving and maintaining the integrity of passageways, tubes or cavities following intrusion. As illustrated, the system includes overlapping degradable sides 214 (Proximal portion) and 216 (Distal portion) delivering at least one active therapeutic 218. In this embodiment, each wrap contains an Adventitial (Proximal) portion 214 and an Interstitial (Distal) portion 216 and the active ingredient is embedded within the Matrix Material in the Adventitial side to provide a unidirectional flow, shown by the arrows towards the tunica interna (intima) portion of the vessel. In another aspect of this invention, the delivered active ingredient continues to reside in the tunica layers of the vessel during the course of treatment.

The antiproliferative agents can be bound or unbound antiproliferative drugs selected from the group including any one of sirolimus, everolimus, zotarolimus, myolimus, corolimus, novolimus, biolimus, paclitaxel, rosiglitazone, pioglitazone, LAB 642, doxyrubicin, aspirin, naprosyn, ibuprofen, diclofenac, estrogen 17-B-estradiol, sildenafil, tadadafil, vardenafil, aminophyline, theophyline, cilostazole, sodium nitroglycerin, sodium nitroprusside, nitroglycerin, isosorbide trinatre, clopidogril, prasugrel, ticagrelor, elinogrel, iloprostane, ximilogatran, dabigatran, atopaxar, apixaban, rivaroxaban, enalapril, lisinopril, candesartan, irbesartan, valsartan, and the like. Some of these drugs are best delivered as free drug from the encircling mesh, sheath or sheet. Others have greater vascular and cellular penetration when carried in either a microparticle structure, liposome or in the form of a nanoparticle. Accordingly as described herein, these drugs can be dispersed, embedded, bound or encapsulated into the Matrix Material in form of a plurality of particles.

As in the previous embodiments, the Interstitial side 216 directs the flow of the at least one active component of the exterior portion coating adventitially, specifically, directed to the enveloping or covering membrane or sheath of body tissue. The device 210, will ensure one-way flow into the adventitial portion of the blood vessel and will have the capacity to hold a quantity of drug sufficient to reach the desired anti-proliferative concentration, whether in free form incorporated onto or into the mesh, sheath or sheet or intercalated into the mesh, sheath, or sheet held in microparticles, liposomes or nanoparticles. When this invention is intact, in place, and delivering drug over a prolonged period of time it will allow the traumatized vessel, either from surgical intervention, balloon dilation or other forms of manipulation, to return to its normal functional state whereby it produces endogenous mediators such as nitric oxide and prostacyclin to block vascular smooth muscle proliferativation with consequent stenosis and occlusion and maintaining patency and perfusion.

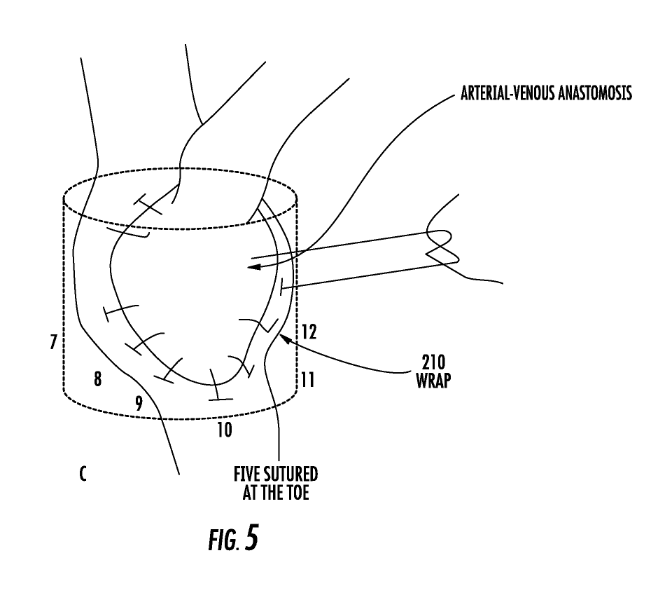

Referring to the FIG. 4, the system 210 is illustrated as a wrap around a coronary artery bypass graft. In accordance to this embodiment, the wrap does not contain an outer PTFE layer that can precipitate an inflammatory process. FIG. 5 is the system 210 at the site of arterial-venous anastomosis. In this embodiment, the system of 210 is a single mesh, sheath or unilayer wrap that is wrapped around the external wall of the vessel of interest.

The specific design of the present invention can vary without deviating from the spirit of the invention. FIGS. 4 and 5 can be adapted to various shapes and sizes customized towards patient's needs. Those of ordinary skill in the art can appreciate the design most beneficial to a subject, depending on the treatment site and intrapatient variability. Accordingly, variations in designs based on subjects needs would not deviate from the core concept of the invention as presented herein.

Another aspect of the present invention is that the innermost Adventitial portion of the wrap degrades faster than the interstitial portions. This design of a completely biodegradable unit would eliminate the flaw in prior art devices with a biodegradable drug-eluting mesh overlaid by a non-biodegradable PTFE coat which, as a result, created a diffusion gradient creating a fluid-filled pocket which acted as a suitable environment for microbial growth and post-surgical infections.

The present invention has addressed such a shortcoming in prior art because the drug containing interior portion of the wrap and the non-drug containing exterior portion are both completely biodegradable with the exterior portion degrading over a longer period of time than the interior layer also ensuring consistent unidirectional flow. As such, these properties of the invention will not allow for the creation of any pocket conducive to microbial invasion or deposition found on and/or within the perivascular surface of the treatment site.

In another embodiment, the system is a sheath pliable and adhesive to conform to the shape of the targeted conduit vessel and stay in place with minimal deformation and constriction of the treated vessel and thus not affecting flow. In this embodiment, the sheath has stability at body temperature (37° C.) maintaining activity for up to 60-120 days but as previously discussed, the material for the system and active components can vary this time period as desired for the particular intended use.

In this embodiment, the sheath is typically in the form of flexible woven, knitted mesh and solid sheets and has a thickness ranging from about 25 microns to about 3000 microns and from about 50 to about 1000 microns from about 100 to 400 microns in thickness and includes fibers that are of the same dimension or of differing dimensions, which can be formed from the same or different types of biodegradable polymers. Furthermore, the presently described system possesses sufficient porosity to permit the flow of fluids through the pores of the Adventitial side.

The active therapeutic component on the interior portion can be applied using various methods such as dip coating, spray coating, solvent casting, extrusion, roll coating, etc. Those of ordinary skill in the art can appreciate that other methods such as physiosorption, chemisorption, ligand/receptor interaction, covalent bonds, hydrogen bonds, ionic bonds, and the like may be employed. As such the therapeutic agent may be attached, bound directly to the matrix fibers or dispersed, embedded or encapsulated therein.

Furthermore, depending on the type of therapeutic or biological agent used and the configuration of the carrier (i.e. film, sheet, open flat mesh, foam, etc.), the present invention can be used for the treatment of various forms of cancer such as primary brain cancer (Glioblastoma Multiforme and Anaplastic Astrocytoma), in situ solid tumors such as ovarian cancer, renal cancer and as found in ductile carcinomas of the pancreas, prostate, breast, and biliary tracts, and lung cancer. Accordingly, the uses of the present systems described herein are not meant to be limiting. Those of ordinary skill in the art can appreciate that the present system can also be applied to patients in need of joint repair, or suffering from eye diseases such as macula edema and diabetic retinopathy, or at risk of heart failure in the form of dilated cardiomyopathy, in gynecological procedures, as post-surgical organ wraps to prevent adhesions, on orthopedic devices, and in chronic obstructive pulmonary disease.

The following prophetic examples further provide the scientific basis for the present invention.

### Example 1

At least one application of the presently described system is in treating patients suffering from peripheral arterial disease (PAD) of the lower leg; in particular, critical limb ischemia (CLI), and severe intermittent claudication. These conditions can potentially lead to leg amputation. As a means of salvaging the limb, such patients will undergo a surgical intervention involving vascular bypass graft surgery. Vascular bypass graft surgery is associated with a high incidence of re-occlusion as the result of restenosis in these grafts leading to limb amputation with a consequent high degree of morbidity and mortality. Placing a system according to the teachings of the present invention around the grafted vessel at the time of implantation can ameliorate future adverse outcomes secondary to restenosis of the vessel.

### Example 2

Those of ordinary skill in the art can appreciate that the same concerns exists in patients suffering from coronary artery bypass graft surgery (CABG). The bypass grafts have a significant incidence of occlusion. Placing a system according to the teachings of the present invention around the CABG in accordance to the present invention will greatly reduce the risk of failure due to restenosis.

### Example 3

In patients with end-stage kidney disease who undergo hemodialysis, the arterial-venous PTFE grafts used as the ports to filter blood through the dialysis apparatus have a high occlusion rate and a short patency life. The occlusion is due to vascular smooth muscle cell proliferative stenosis at the venous site of the anastomotic connection between the PTFE graft and the vein. Placing the instantly described system at this site at the time of implantation or when the occluded graft is balloon dilated will prolong graft patency and thereby prolonging patient survival thus increasing the patient's probability of receiving a kidney transplant.

Arterial-venous fistulas have recently gained more attention over PTFE grafts among practioners for patients in need of hemodialysis because such an avenue of access remains patent significantly longer than PTFE grafts. However, there is still an approximately 30% rate of failure in maturation of such vascular devices which means only 70% become functional. Additionally, these devices can occlude during patient's treatment course. The present inventor has addressed this problem in the art, as placing the presently described system on the fistula at the time of its creation can significantly increase maturation rates and prolong patient survival.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. A number of embodiments of the invention have been described. Nevertheless, it will be understood that various modifications may be made without departing from the spirit and scope of the invention. Accordingly, other embodiments are within the scope of the following claims.

1. An implantable, anti-proliferative agent-containing perivascular system adapted to be placed in contact with the exterior wall of a vascular structure comprising an interior and exterior side, wherein the interior side comprises an adhesive, flexible, biodegradable matrix material and an antiproliferative agent, wherein the antiproliferative agent is released from the matrix substantially following a net unidirectional flow pattern.

2. The system of claim 1, wherein said system is a circumvascular wrap formed from a mesh, a foam, a gel, sheet, or shealth.

3. The system of claim 1, wherein the system is a unilayer unit.

4. The system of claim 1, wherein the antiproliferative agent is selected from the group consisting of sirolimus, everolimus, biolimus, zotarolimus, corolimus, myolimus, paclitaxel, taxanes, tacrolimus, dexamethasone, steroids, fractionated heparin, unfractionated heparin, metalloproteinase inhibitors, factor Xa inhibitors, direct thrombin inhibitors, PAR 1 and 4 antagonists, flavoperidol, angiotensin converting enzyme inhibitors such as enalapril and lisinopril, angiotensin receptor anatagonists such as irbesartan, candesartan and valsartan, human bone marrow cells, stem cells, genetically modified human cells, glycoprotein IIB/IIIA antagonists, P2Y₁₂ antagonists, antibiotics and any functional derivatives thereof.

5. The system of claim 1, wherein the matrix material is biodegradable and comprises at least one compound selected from the group consisting of polymers or copolymers containing omega-3 or omega-6 fatty acids, fish oil, vitamin E, polylysine, polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, chitin, chitosan, gelatin, collagen, hyaluronic acid, fibrin, and copolymers, or mixtures thereof.

6. The system of claim 5, wherein the matrix material is a polymer or copolymer containing omega-3 or omega-6 fatty acids.

7. The system of claim 1, wherein the vascular site of interest is selected from the group consisting of neoplastic regions and anastomotic sites of an arterial-venous synthetic graft and graft composed of biologic material as found in kidney hemodialysis, peripheral vascular bypass graft surgery, and coronary artery bypass graft surgery.

8. The system of claim 1, wherein the matrix is substantially free of non-biodegradable polymers.

9. The system of claim 8, wherein the matrix is free of PTFE.

10. The system of claim 1, wherein the unidirectional flow of the therapeutic agent is from exterior side of the system towards the interior side of the system.

11. A method of treating a patient at risk of developing vascular stenosis comprising the steps of: locally administering a biodegradable drug eluting system comprising an interior and exterior side, wherein the interior (adventitial) side comprises an adhesive flexible, biodegradable matrix material and an anti-proliferative agent, wherein the antiproliferative agent is released from the matrix substantially following a net unidirectional flow pattern towards the perivascular region of the vascular structure, and (b) delivering an active agent into the vascular wall.

12. The method of claim 11, wherein system is wrapped around the exterior borders of the site of interest.

13. The method of claim 11, wherein the antiproliferative agent is selected from the group consisting of sirolimus, everolimus, biolimus, zotarolimus, corolimus, myolimus, paclitaxel, taxanes, tacrolimus, actinomycin D, dexamethasone, steroids, fractionated heparin, unfractionated heparin, fondaparinux, factor Xa inhibitors, direct thrombin inhibitors, PAR 1 and 4 antagonists, metalloproteinase inhibitors, flavoperidol, angiotensin converting enzyme inhibitors, angiotensin receptor antagonists, human bone marrow cells, stem cells, genetically modified human cells, glycoprotein IIB/IIIA antagonists, antibiotics and any combinations thereof

14. A method of claim 9, wherein the biodegradable drug eluting system is an implantable mesh, an implantable matrix, a foam, a gel or a coating formulation.

15. A method of claim 11, wherein the system stays intact in vivo for at least 4 months.

16. The method of claim 11, wherein the matrix material is biodegradable and comprises at least one compound selected from the group consisting of polymers or copolymers containing omega-3 or omega-6 fatty acids, fish oil, vitamin E, polylysine, polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, chitin, chitosan, gelatin, collagen, hyaluronic acid, fibrin, and copolymers, or mixtures.

17. The method of claim 16, wherein the matrix material is a polymer or copolymer containing omega-3 or omega-6 fatty acids.

18. A kit for preserving the patency of a vascular structure comprising an implantable, anti-proliferative agent-containing perivascular system adapted to be placed in contact with the exterior wall of a vascular structure comprising an interior and exterior side, wherein the interior side comprises an adhesive, flexible, biodegradable matrix material and an anti-proliferative agent, wherein the antiproliferative agent is released from the matrix substantially following a net unidirectional flow pattern.

19. The kit of claim 18, wherein said system in the form of a circumvascular wrap derived from a mesh, a foam, a gel or a shealth.

20. The kit of claim 18, wherein the system is a unilayer unit.

21. The kit of claim 18, wherein the antiproliferative agent is selected from the group consisting of sirolimus, everolimus, biolimus, zotarolimus, corolimus, myolimus, novolimus, paclitaxel, taxanes, tacrolimus, dexamethasone, steroids, fractionated heparin, unfractionated heparin, fondaparinux, matrix metalloproteinase inhibitors, angiotensin converting enzyme inhibitors, angiotensin receptor antagonists, flavoperidol, human bone marrow cells, stem cells, genetically modified human cells, glycoprotein IIB/IIIA antagonists, factor Xa inhibitors, direct thrombin inhibitors, PAR 1 and 4 antagonists, antibiotics and any functional derivatives thereof.

22. The kit of claim 21, wherein the matrix material is biodegradable and comprises at least one compound selected from the group consisting of polymers or copolymers containing omega-3 or omega-6 fatty acids, fish oil, vitamin E, polylysine, polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, chitin, chitosan, gelatin, collagen, hyaluronic acid, fibrin, and copolymers, or mixtures thereof.

23. The kit of claim 22, wherein the matrix material is a polymer or copolymer containing omega-3 or omega-6 fatty acids.
